(19) United States
(12) Patent Application Publication
(10) Pub. No.: US 2008/0003264 A1
(43) Pub. Date: Jan. 3, 2008

(54) Confectionary product for expressing and controlling the emotions of individuals

(76) Inventor: Judy L. Cerano, White Lake, MI (US)

Correspondence Address: KNECHTEL, DEMEUR & SAMLAN, 525 W. MONROE STREET, SUITE 2360, CHICAGO, IL, 60661, US

(21) Appl. No.: 11/476,283

(22) Filed: Jun. 28, 2006

(51) Int. Cl. A61K 47/00

(52) U.S. Cl. 424/439; 426/660

(57) ABSTRACT

A confection product invention comprising a plate made of candy, a covering for receiving the candy plate, and a means for enclosing the candy plate within the covering. A stand is also provided for holding the confection product until it is needed. The method for using the confection product to express or control the emotions of individuals comprises at least throwing the confection product against a hard surface such as a wall or floor for breaking the candy plate into pieces, opening the covering to expose the pieces of the broken candy plate, retrieving a piece of the broken candy plate, and eating the piece of the candy plate.

### I. FIELD OF THE INVENTION

The present invention relates to confectionary products and, more particularly, to a confectionary product for expressing and/or controlling the emotional disposition or state of individuals.

### II. DESCRIPTION OF THE PRIOR ART

All across the world billions of individuals are living their daily lives as best they can and acting or reacting to the activities or events that they are experiencing. Such activities or events include but are not limited to working, schooling, traveling, vacationing, socializing, playing, resting, driving, running, biking, partying, dining, entertaining, etc. . . . The list is endless. The commonality with all of these activities or events is that the activities or events cause each individual to experience a certain emotion or any spectrum of emotions which must be controlled by the individual. Absent such control, the emotions can have an undesirable impact on the individual or any persons related to, having any connection with, and/or in close proximity to the individual.

Thus, there is a need and there has never been disclosed Applicant's unique confectionary product for controlling the emotional disposition or state of individuals.

### III. OBJECTS OF THE INVENTION

It is the primary object of the present invention to provide a confectionary product and means for expressing, controlling, and/or improving the emotions of individuals. A related object of the present invention is to provide a confectionary product for an individual to release or expel their negative emotions. A further related object of the present invention is to provide a confectionary product for an individual to enhance their positive emotions.

Another object of the present invention is to provide a means for limiting any undesirable actions taken by individuals in connection with or relating to any emotions that they are experiencing.

Another object of the invention is to provide a confectionary product that rewards an individual for their actions in using the confectionary product for controlling or improving their emotions. A related object of the present invention is to provide a confectionary product that is made of a candy material having such a variety, types, and flavoring that it is desirable to be used by the individuals.

Still another object of the invention is to provide a product that, if used properly, is safe and easy to use.

Other objects of the present invention will become more apparent to persons having ordinary skill in the art to which the present invention pertains from the following description taken in conjunction with the accompanying drawings.

### IV. SUMMARY OF THE INVENTION

The present confectionary product invention comprises a plate made of candy, a covering for receiving the candy plate, and a twist bow tie for enclosing the candy plate within the covering. A stand is also provided for holding the confectionary product until it is needed. The method for using the confectionary product to express, control, or improve the emotions of individuals comprises at least throwing the confectionary product against a hard surface such as a wall or floor for breaking the candy plate into pieces, opening the covering to expose the various pieces of the broken candy plate, retrieving a piece of the broken candy plate, and eating the piece of the candy plate.

### V. BRIEF DESCRIPTION OF THE DRAWINGS

The Description of the Preferred Embodiment will be better understood with reference to the following figures:

FIG. 1 is a perspective view of Applicant's confectionary product invention and, in particular, illustrating a candy plate being received into a covering which will be secured by a twisting bow tie.

FIG. 2 is a side view of Applicant's confectionary product invention being thrown against a wall or other solid surface resulting in the candy plate being broken into pieces and remaining contained within the covering.

FIG. 3 is a side view of Applicant's confectionary product invention as the covering is being opened for exposing access to the broken pieces of the candy plate.

FIG. 4 is a side view of a person eating a broken piece of the candy plate.

### VI. DETAILED DESCRIPTION OF THE PREFERRED EMBODIMENT

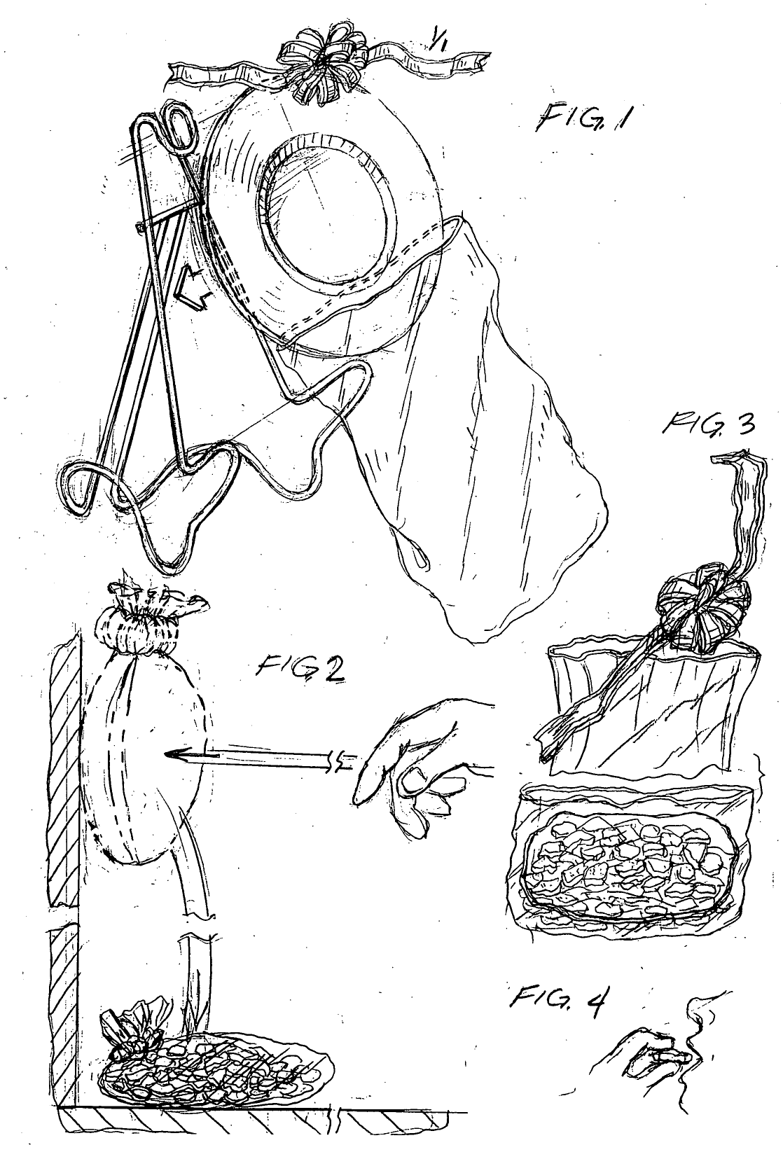

Turning first to FIG. 1, Applicant's confectionary product 8 is illustrated. The confectionary product 8 comprises a confection 10, a covering 12, and a bow tie 14.

In the preferred embodiment, the entire confection 10 is made of an edible, candy material. Alternatively, it is contemplated that any portion of the confection 10, less than the entire confection 10, may be made of the edible, candy material provided that the confection 10 is made of a sufficient amount of candy to accomplish the intended purpose of the invention as disclosed herein. In this alternate embodiment, the remaining portion of the confection 10 not made of the edible candy can be made of any other type of material that is known to one skilled in the art and that is adaptable to be used in combination with the portion of the confection 10 that is made of candy for forming the confection 10.

The candy material of the confection 10 preferably comprises any combination of ingredients for creating the candy; provided that, when completed, the candy is sufficiently rigid such that it may be broken into pieces as described later in the specification. Alternatively, it is contemplated that the candy, when completed, may provide any variation from flexibility to rigidness as is available and known to one skilled in the art. It is further contemplated that the candy may be enhanced with any flavoring including but not limited to cherry, root beer, pina colada, banana cream, grape, blueberry, wintergreen, strawberry, or any other available flavoring known to one skilled in the art.

The confection 10 can formed into any design, shape, or pattern as is desired and available for making candy or any combination of candy with other materials. In the preferred embodiment and as illustrated in FIG. 1, the confection 10 is made into a plate design. Alternatively, it is contemplated that the design or shape of the confection 10 may be made into any other type of product including but not limited to any kitchen product such as a glass, cup, mug, silverware; any type of household or consumer type product; any type of character; or any other fanciful or arbitrary type of design or shape.

The covering 12 preferably comprises an enclosed body 16 having an opening 18. In the preferred embodiment, the body 16 has a width 20 that is substantially equal to the width of the confection 10. Likewise, the opening 18 is designed to extend across the entire width 20 of the body 16 for enabling the confection 10 to be easily inserted through the opening 18 and directly into the body 16 without causing any problems.

In the preferred embodiment, the covering 12 is made of a cellophane material. Alternatively, it is contemplated that the covering 12 may be made of a suitable plastic film or any other type of material known to one skilled in the art provided that the material is sufficient to accomplish the purpose of the covering 12 provided below. The covering 12 is also preferably made of a transparent material such that the candy confection 10, or any portion thereof, is made visible through the covering 12.

The purpose of the covering 12 is: (1) to completely cover and retain the confection 10 within the covering 12; (2) to be made of a material sufficiently durable to withstand an impact with a rigid object such as a wall or floor without tearing or exposing any openings or holes in the covering 12 thereby continuing to retain the entire confection 10 therein; (3) to be made of a material sufficiently durable to withstand the confection 10 being broken into pieces when engaging the rigid object without tearing or exposing any openings or holes in the covering 12 (i.e., even with some broken pieces of the confection 10 presumably resulting in sharp edges). This enables the covering 12 to prohibit any of the confection 10 from being uncontrollably thrown from the covering 12 which could cause injury or harm to others; and (4) to be made of a transparent material enabling the individual using the candy confection 10 to visually see the candy confection 10 actually engaging the rigid object and being broken into pieces as a result of the impact.

Once the candy confection 10 is received into the covering 12, the bow tie 14 is used for twisting around and closing the opening 18 thereby securing the candy confection 10 within the covering 12. Alternatively, the means for closing the opening 18 and securing the candy confection 10 within the covering 12 includes but is not limited to hook and loop fasteners such as Velcro®, rope, wire, twine, clasp, staples, stitching, glue, fusion or other form of bonding, and/or any other means known to one skilled in the art.

Alternatively, it is contemplated that the confectionary product 8 may be packaged in a shrink wrap or other form fitting covering 12 thereby eliminating the necessity of an opening 18 in the body 16 to receive the confection 10 and/or a bow tie 14 to close the opening 18.

Should it be desirable, a stand 22 is provided to store and display the confectionary product 8 until the confectionary product 8 is to be used.

When the confectionary product 8 is to be used, an individual locates the confectionary product 8, which may or may not be displayed in the stand 22, and takes possession of the confectionary product 8 such as in their hand 24. In a non-limiting example as illustrated in FIG. 2, the individual then sets the confectionary product 8 in motion by throwing the confectionary product 8 toward a hard or rigid object or surface 26. In the preferred embodiment, the hard or rigid object or surface 26 is an immovable object such as a wall, floor, or any other object that is sufficiently firm to be unaltered by the impact.

When the confectionary product 8 comes in contact with the hard or rigid object or surface 26, the impact causes the confection 10 to break into various pieces. If the confectionary product 8 engages a wall as illustrated in FIG. 2, the confection 10 may further break into additional pieces 28 when the confectionary product 8 falls and comes in contact with the floor. Alternatively, it is contemplated that any other means for breaking the confection 10 into various pieces can be used including but not limited to throwing the confectionary product 8 against a movable hard or rigid object or surface, striking the confectionary product 8 with a solid object, or any other means known to one skilled in the art and/or the imagination or ingenuity of the individual.

When the confectionary product 8 is no longer in motion, the individual retrieves the confectionary product 8 and removes the bow tie 14 from the confectionary product 8. This releases the opening 18 in the covering 12 exposing the various pieces 28 of the confection 10, as illustrated in FIG. 3. Alternatively, the individual may expose the various pieces 28 of the confection 10 by creating a hole 30 in the covering 12, also illustrated in FIG. 3. This alternate means for exposing the various pieces of the confection 10 includes but is not limited to cutting an opening in the covering 10 with scissors or the like, puncturing a hole in the covering 10 using the individual's finger or other puncturing device, or any other means known to one skilled in the art. With the various pieces 28 of the confection 10 exposed, the individual reaches in through the opening 18 or hole 30 to grab a piece 32 of the confection 10 and then eat it, as illustrated in FIG. 4.

Alternatively, it is contemplated that the confectionary product 8 may comprise a confection 10 that is made of a malleable material such that the confection 10 is immune from being broken into various pieces. One example would be a rubbery textured confectionery commonly known as gummy bears.

As everyone experiences various feelings that accompany positive or negative emotions, any of these emotions can be controlled or improved by using the confectionary product 8 as disclosed above. Although the list of positive or negative emotions is endless, some of the emotions include exuberance, joy, happiness, anger, rage, frustration, disappointment, etc. . . . Since dealing with such emotions is important to one's health (e.g., repression of negative emotions can lead to depression, anxieties, panic, eating disorders, etc. . . . ) and the health and safety of others, Applicant's confectionary product 8 provides the solution.

When an individual experiences and begins to express any emotion that desires to control or improve, the individual can turn to and focus on Applicant's confectionary product 8. This initial interest distracts the individual's attention away other outlets such as another person or thing. With the confectionary product 8 in the individual's possession and the individual still experiencing the emotions, the individual can exert or use these emotions into the energy exerted for throwing the confectionary product 8 toward a hard or rigid object or surface 26. When the confectionary product 8 impacts the hard or rigid object or surface 26, the individual observes the impact and the confection 10 within the covering 12 breaking into the various pieces 28. Alternatively, the individual may hear the audible sounds of the confection 10 breaking into the various pieces. This resulting action of the confection 10 breaking and the visual or sound recognition of the action further assists the individual to release and expel negative emotions or enhance the positive emotions. The subsequent retrieval of the confectionary product 8, opening of the covering 12 to expose the various pieces 28, and eating any of the various pieces 28 of the confection 10 essentially rewards the individual for their actions in using the confectionary product 8 and limiting any of the possible actions available to the individual as a result of these emotions to be confined to the confectionary product 8 only. The preferred candy embodiment of the confection 10 and the various types and flavors available provide a confectionary product 8 that is desirable to any individual. In this manner, the confectionary product 8 is used for controlling the emotions of said individuals.

If the confectionary product 8 is made of a malleable material, the confection 10 will not break into various pieces. However, the confection 10 could be deformed, bounce off the hard or rigid object or surface 26, or have reactions other than breaking which can have the same affect on the individual as the breaking.

Alternatively, the confectionary product 8 may be used to continue the historic tradition known as Kabbalat Panim where, during a wedding reception, the mothers of the bride and groom stand together and break a plate as a sign of the bride and groom's commitment to one another.

Thus, there has been provided a unique confection product for controlling or improving the emotions of individuals. While the invention has been described in conjunction with a specific embodiment, it is evident that many alternatives, modifications and variations will be apparent to those skilled in the art in light of the foregoing description. Accordingly, it is intended to embrace all such alternatives, modifications and variations as fall within the spirit and scope of the appended claims.

What is claimed is:

1. A method for improving the emotional disposition of an individual, comprising the steps of:
providing an edible confectionary into a flexible, clear plastic container;
giving the container to the individual with the emotional disposition to be improved;
directing the individual to throw the container against a rigid surface sufficient to cause the edible confectionary to break into various pieces;
allowing the individual to throw the container against the rigid surface;
having the individual eat at least one of the various pieces of the edible confectionary; and
whereby the emotional disposition of the individual is improved.

2. The method of claim 1 further including the step of providing an edible confectionary that is made of candy.

3. The method of claim 1 further including the step of providing a flexible plastic container that is made of a cellophane material.

4. The method of claim 1 further including the step of providing a clear plastic container that is transparent enabling the edible confectionary therein to be visually seen by the individual.

5. The method of claim 1 further including the step of securing the edible confectionary within the container.

6. The method of claim 1 further including the step of identifying the individual with the emotional disposition to be improved, the emotional disposition comprising a positive or negative emotion.

7. The method of claim 1 further including the step of witnessing the confectionary breaking into various pieces.

8. The method of claim 1 further including the step of flavoring the edible confectionary.

9. The method of claim 1 further including the step of removing the at least one of the various pieces of the edible confectionary from the container.

10. The method of claim 8 further including the step of enjoying the flavoring of the edible confectionary.

11. A method for controlling a user's emotional state, comprising the steps of:
placing an edible confectionary in a flexible, transparent, pouch;
grasping the pouch by the user;
propelling the pouch against a rigid surface causing the edible confectionary to change;
witnessing the changing of the confectionary through the transparent pouch;
eating at least a portion of the confectionary; and
whereby the user's emotional state is controlled.

12. The method of claim 11 further including the step of securing the edible confectionary within the pouch.

13. The method of claim 10 wherein the step of propelling the pouch against a rigid surface causing the edible confectionary to change further comprising the step of the edible confectionary breaking into various pieces.

14. The method of claim 11 wherein the step of propelling the pouch against a rigid surface causing the edible confectionary to change further comprising the step of the edible confectionary deforming due to the impact.

15. The method of claim 11 further including the step of removing the at least a portion of the confectionary from the pouch.

16. A method for improving the emotional state of an individual, comprising the steps of:
placing an edible confectionary in a flexible, transparent receptacle;
securing the confectionary within the receptacle;
taking possession of the receptacle;
causing the edible confectionary to break inside the receptacle;
witnessing the breaking of the confectionary through the transparent receptacle;
removing at least a portion of the confectionary from the receptacle;
eating the at least a portion of the confectionary removed from the receptacle; and
whereby the emotional state of the individual is improved.

17. The method of claim 16 wherein the step of causing the edible confectionary to break inside the receptacle further comprising the step of throwing the receptacle against a rigid surface.

18. The method of claim 16 wherein the step of causing the edible confectionary to break inside the receptacle further comprising the step of striking the receptacle with an object.
